(19) United States
(12) Patent Application Publication
(10) Pub. No.: US 2015/0323806 A1
(43) Pub. Date: Nov. 12, 2015

(54) Eyeglass and medical device retainer and sensor

(71) Applicant: Christianne Elizabeth Arroyo, Palmetto Bay, FL (US)

(72) Inventor: Christianne Elizabeth Arroyo, Palmetto Bay, FL (US)

(21) Appl. No.: 14/270,493

(22) Filed: May 6, 2014

(51) Int. Cl. G02C 3/00; A61M 16/06; G08B 21/04; A61B 5/0205; G01P 15/02; A41D 20/00; A61M 16/00

(52) U.S. Cl. CPC G02C 3/003; A41D 20/00; A61M 16/0672; A61M 16/0683; A61M 16/0003; A61B 5/02055; G01P 15/02; G08B 21/0446; G08B 21/0453; A61M 2202/0208; A61M 2205/586; A61M 2230/50; A61M 2230/06; A61M 2230/63; A61M 2205/3553

(57) ABSTRACT

An eyeglass and medical device retainer device, to hold a wearer's glasses or medical devices in place is disclosed. In one preferred embodiment, the device is an adjustable headband worn around the circumference of the wearer's head, and having at least one loop element to wrap around the eyeglasses and hold them place. In another preferred embodiment, the device is an adjustable headband worn around the circumference of a patient's head, and having at least one loop element to wrap around a medical device to be held in position on the patient's head. Such medical devices to be held may include oxygen tubes that are placed in proximity to a patient's nose or nostrils. In other preferred medical application embodiments, the head band further comprises sensors to monitor patient body temperature, patient pulse, and to monitor for inadvertent falls.

### FIELD OF THE INVENTION

The present invention generally relates to eyeglass and medical device retainers, and more particularly relates to a headband type device that holds eyeglasses or medical equipment in place on the user's or patient's head using at least one loop element as the means for retaining the eyeglasses or medical equipment in place. In preferred embodiments, the retainer also comprises sensor elements to monitor a patient's temperature and pulse. In still further embodiments, the retainer can also include one or more accelerometers to monitor rapid movements and be used as a fall detector.

### BACKGROUND OF THE INVENTION

At some point in most everyone's life, they will need to use eyeglasses or sunglasses. Eye protection from the sun has become a necessity given the medically know concern for protection of our eyes from ultraviolet (“UV”) rays. As we age, most individuals find a need for wearing “reader” glasses in order to see objects up close or simply for reading. Moreover, eyeglasses are used by people in every possible occupation and activity, and all walks of life. Indeed, professionals who need precise and clear vision, including doctors, aircraft pilots, automobile race drivers, golfers, tennis players, runners, and other athletes often have the need to use glasses to properly accomplish their jobs, tasks or activities. However, if the user's glasses slide down their nose, or otherwise move or are lodge out of position, the wearer can no longer see properly and must reposition the glasses.

Although contact lenses and surgical corrective vision is becoming more prevalent for people needing vision correction, even with such corrections through contact lenses or laser surgery, people will nonetheless still need to wear sunglasses when they are outside to protect their eyes from UV rays, or need to wear “reader” glasses for correct close up vision.

In addition to their occupations, people also wear eyeglasses during all types of recreational activities including running, biking, basketball, baseball, soccer, skiing and racquet sports. It is during these type of recreational activities that a major nuisance with the use of eyeglasses emerges. Glasses slide down or move down a wearer's nose, especially when the wearer perspires or sweats. Because glasses provide vision correction only when worn in the proper location, when the wearer's glasses slide down or move, their vision is no longer properly corrected. The wearer is then compelled to push the glasses back up into position. This cycle of the glasses sliding down and the wearer pushing the glasses back up is repeated all too frequently.

Similarly, in the medical or hospital setting, a patient is often required to have certain medical devices located near or on his or her head. One primary example is the use of oxygen tubes to provide oxygen directly to the nose or nostrils of the patient. Such tubes are often time taped directly to the patient's head to maintain the tubes in position. The use of tape on a patient's head or facial area can be uncomfortable, especially when the tape is to be removed.

Several examples of eyeglass retainer devices have been designed and patented. One example, by the present inventor, is for an eyeglass retainer, and is shown and described in U.S. Pat. No. D374,114. The '114 design patent is limited to the particular configuration, shape and construction of the head band and loop element. The '114 design does not disclose the use of multiple loop elements, and does not disclose or suggest the use of the head band to hold any medical devices or to incorporate sensing technology for temperature, pulse, heartrate, or accelerations.

What is needed is a simple and comfortable device that can be easily attached to the user's eyeglasses, sun glasses, or to medical devices, such as oxygen tubes, and is easily adjustable to each wearer's desired configuration or size, and can maintain the position of the glasses or medical devices on the user's head.

### SUMMARY OF THE INVENTION

The above noted problems inadequately or incompletely resolved by the prior art are addressed and resolved by the present invention.

A preferred embodiment of the invention is an eyeglass retainer to be worn with eyeglasses, comprising a head band worn around the circumference of a wearer's head, looping means having two ends such that the first end is attached to the headband and the second end wrapping around the eyeglasses, attaching means incorporated into the second end of the looping means such that the second end of the looping means is removably attachable to the headband and thereby maintaining the eyeglasses in the position desired by the wearer.

Another preferred embodiment of the invention is as a medical device retainer, comprising a head band worn around the circumference of a patient's head; at least one loop element having two ends such that said first end is attached to the headband and said second end wraps around said medical device to be held in position on a wearer's head; attaching means incorporated into said second end of said at least one loop element such that said second end of said at least one loop element is removably attachable to said headband; thereby maintaining said medical device in a position necessary for proper medical use.

The invention will be best understood by reading the following detailed description of the preferred embodiments in conjunction with the drawings briefly described below.

### BRIEF DESCRIPTION OF THE DRAWINGS

For the purposes of illustrating the invention, the attached drawings show several embodiments that are presently preferred. However, it should be understood that the invention is not limited to the precise arrangement and instrumentality shown in the accompanying drawings.

FIG. 1A: is a perspective view of an exemplary embodiment of the eyeglass holder as claimed herein using a single loop;

FIG. 1B: is a perspective view of an exemplary embodiment of the eyeglass holder as claimed herein using a plurality of retaining loops;

FIG. 2: is a perspective view of an exemplary embodiment of the medical device holder as claimed herein shown holding oxygen tubes in place;

FIG. 3: is a front view of an exemplary embodiment of the medical device holder as claimed herein showing temperature sensors to sense a patient's forehead temperature;

FIG. 4: is a side view of an exemplary embodiment of the medical device holder as claimed herein showing a pulse sensor near a patient's temple; and

FIG. 5: is a front view of an exemplary embodiment of the medical device holder as claimed herein showing an accelerometer to monitor for inadvertent falls, and a transmitter to provide a wireless alarm to one or more recipients.

### DETAILED DESCRIPTION OF THE INVENTION

The present invention is an eyeglass and medical device retainer comprising an adjustable head band, and at least one loop element that wraps around the eyeglasses or medical device being used by the wearer and thereby maintaining the eyeglasses or medical device in the desired position on the wearer's head.

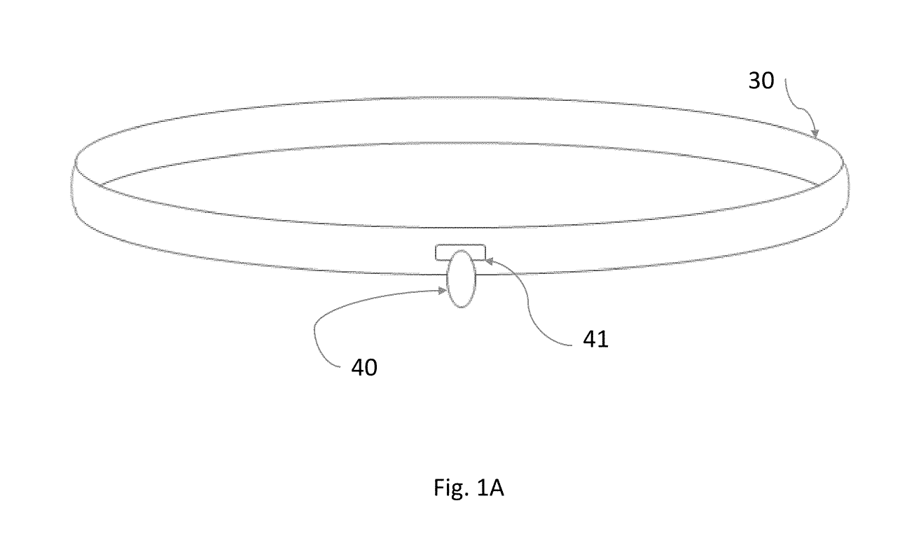

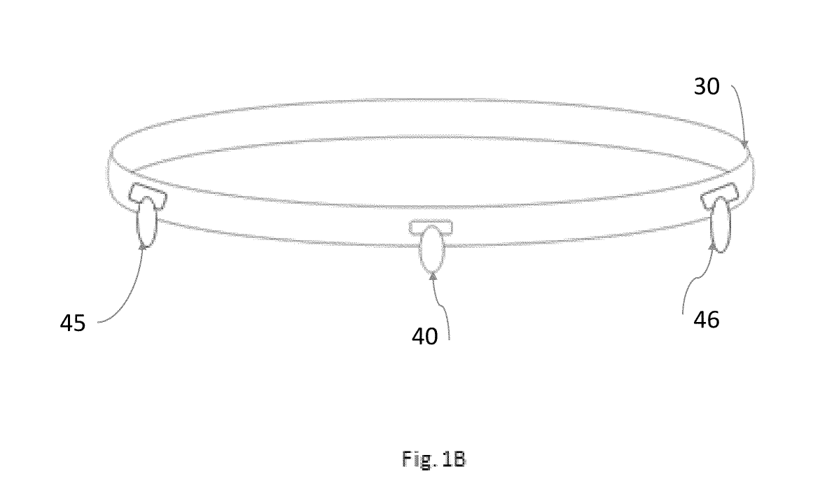

In more particularity, as shown in FIG. 1A, the eyeglass retainer comprises a textile head band 30 with a loop 40. The textile head band 30 is designed to encircle the wearer's head above the wearer's ears and along the forehead area of the wearer. The loop 40 has two ends such that one end is affixed to the head band 30 and the other end is removably attachable to the head band 30. As shown in use, the wearer can use the loop 40 to encircle the center section of the eyeglasses or sun glasses, and then put the head band 30 on along with the eyeglasses or sun glasses. With the user wearing the head band, his or her glasses will be held in position, and will not slide down the wearer's nose due to moisture, sweat, or simply movement.

The second end of the loop may be attached to the head band 30 by means of a snap mechanism, or a button 41. Allowing for complete variability with respect to the position of the glasses on the wearer's head, an alternative means for attaching the loop 40 is through a hook and loop textile, use as Velcro®.

The head band 30 can be manufactured from an elastic textile, such as Lycra®, allowing for variation in size to account for differing circumference dimensions for differing users. Alternatively, the head band 30 can be manufactured with two ends having attachment means at each end. By way of example, the two ends could have respective snaps elements, or a button and hole, or similar to the loop attachment described above, the ends of the head band 30 could have respective hook and loop elements to allow full variability in sizing the head band to the wearer.

In an alternative embodiment of the head band eyeglass holder, FIG. 1B shows head band 30 having a plurality of loop elements 40, 45, 46. As shown, in this embodiment, one loop element 40 can be located in the approximate center of the head band 30, to loop around the center nose piece of the wearer's glasses, and other loop elements 45, 46 can be located on approximate opposite sides of the head band 30, to loop around the respective ear pieces of the wearer's glasses. The loop attachment means for this embodiment can include the same type of connections as described above for the FIG. 1A embodiment, including snaps, buttons, or a textile hook and loop material. The FIG. 1B embodiment provides additional retention and holding points as compared to the FIG. 1A embodiment, and accordingly, will hold the wearer's glasses in position more robustly.

As a head band which may be used by athletes or other workers, the head band 30 can be made of a moisture absorbing material thereby eliminating sweat from getting into the wearer's eyes, or running onto the wearer's glasses. Such head band 30 material should be washable to allow the wearer to keep the device clean. One example of such material could be a terry cloth type material.

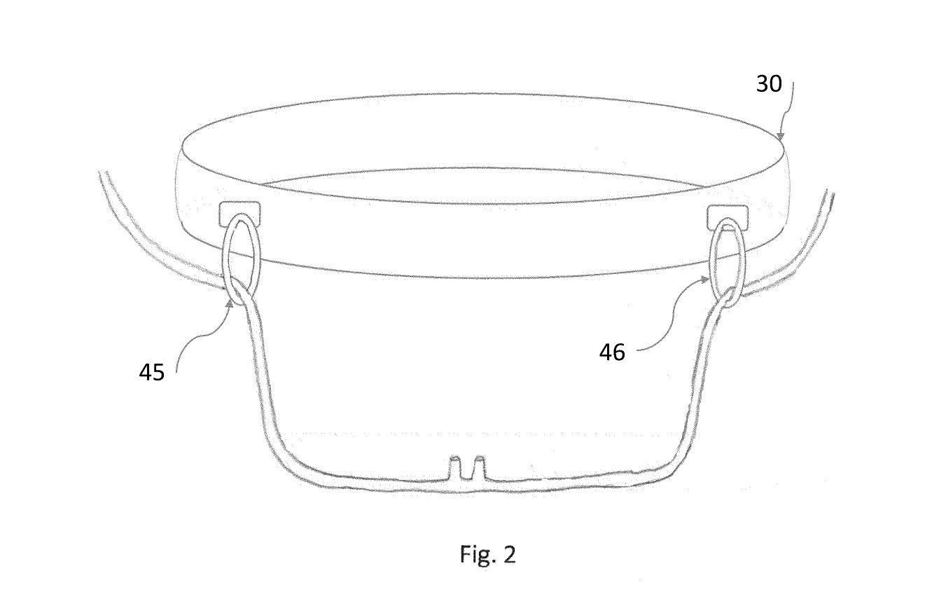

The head band/loop device can, in another preferred embodiment be used by medical patients to hold certain medical devices in place near or on the patient's head. By way of one example, as illustrated in FIG. 2, the head band 30 can be manufactured with a plurality of loops 45, 46 placed along the sides of the head band 30. In such locations, the loops can hold oxygen tubes that need to be held in place near the patient's nostril. Although two loops 45, 46 are shown in FIG. 2, further loops could be used to further hold the oxygen tubes in place. While tape is most often used to hold such tubes in place, the adhesive side of the tape will often cause discomfort to the patient, especially when the tape is to be removed from the patient's head. Where the oxygen tubes are moved or replaced repeatedly, the skin abrasion and discomfort to the patient with repeated removal and replacement of the tape, is only increased and made worse. The head band/loop device as illustrated in FIG. 2 provides a ready solution to this problem.

Similar to the eyeglass retainer embodiment, the medical device holder embodiment may have the head band 30 manufactured from an elastic material, or it may be made with two ends allowing for variable sizing for different patients. For the medical embodiment, the head band 30 materials can be manufactured of a sterile material, especially if the patient may have abrasions or wounds in the general area where the head band 30 encircles the patient's head. Whether the head band 30 is manufactured from a sterile material, or some other textile, for the medical embodiment, the head band/medical device holder should be disposable after each use.

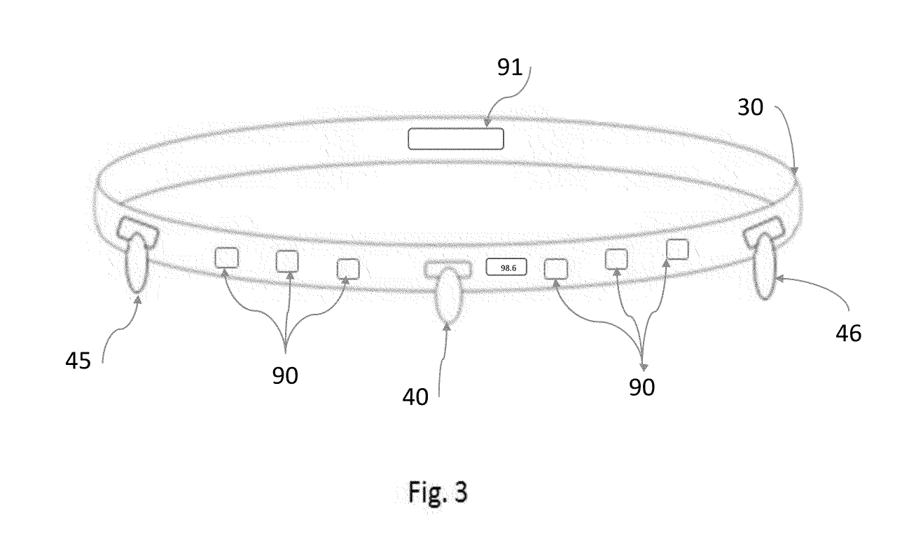

In view of the placement of the head band 30 around a patient's forehead and temple region, in a further preferred embodiment to provide added medical assistance and information to caregivers, temperature sensors 90 can be incorporated along the inside of the head band 30, as illustrated in FIG. 3. Using a plurality of temperature sensors 90 allows for multiple readings to enhance accuracy of the measurement. The temperature sensors 90 could be directly connected to a display incorporated into the head band 30 (as shown in FIG. 3), or the sensors 90 could transmit the temperature data to a transmitter 91 built into the head band 30, which sends a wireless signal to one or more recipients, thereby providing the patient's temperature without the need to disturb the patient during rest.

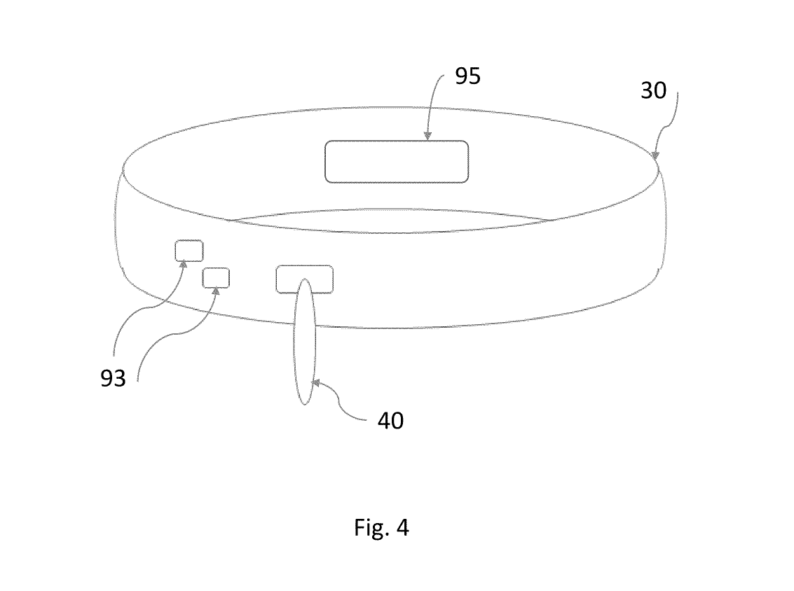

A further enhancement to the medical embodiment of the head band 30 includes, as shown in FIG. 4, a pulse monitor 93 incorporated into the inside of the head band 30 near the temple regions of the patient. Using dual pulse monitors 93 provides some redundancy by reading the patient's pulse at both the left and right hand sides of the patient's head. The pulse monitors 93 provide real-time data to a transmitter 95 embedded into the head band 30. Similar to the above temperature sensor embodiment, the transmitter 95 sends a wireless signal to one or more recipients, which could include a digital or graphical readout in proximity to the patient. Through use of the medical head band 30, caregivers and medical technicians can monitor in real-time the pulse of a patient without further disturbing the patient who may be sleeping or otherwise resting.

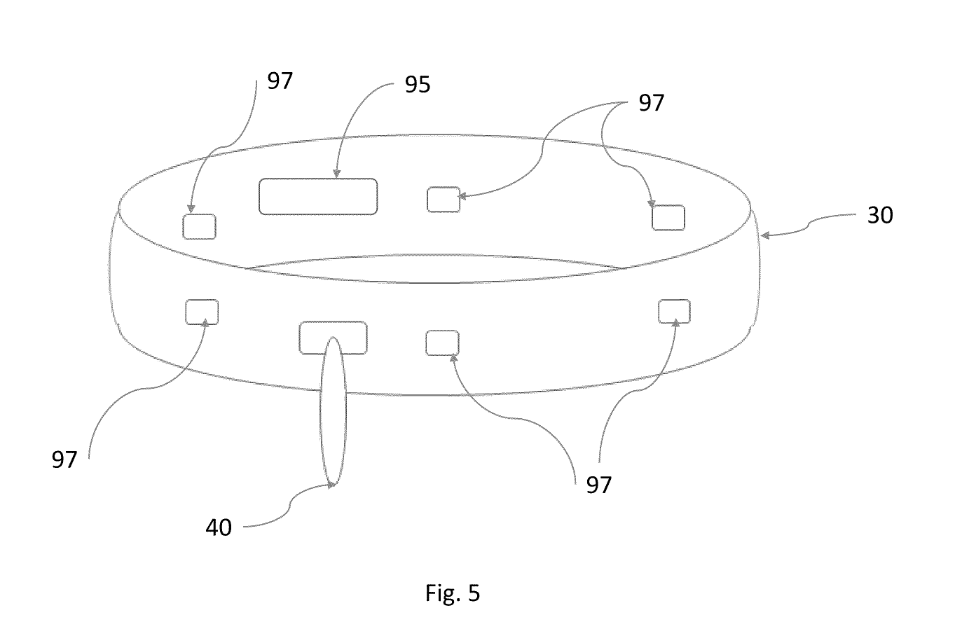

In still a further enhancement to the medical head band device, to monitor for falls or rapid movements of the patient, especially for older patients, at least one accelerometer 97 can be incorporated into the head band 30, as illustrated in FIG. 5. Similar to the FIG. 4 preferred embodiment, the accelerometer, or plurality of accelerometers 97 is connected to a transmitter 95 that can send a wireless signal to one or more recipients. The accelerometers 97 measure rapid movements of the patient's head, and accordingly monitors if the patient were to fall or had a seizure. A plurality of accelerometers can be used for redundancy and also for consistency of sensed accelerations to alleviate potential false alarms should a single accelerometer inadvertently sense a fall. The recipient or recipients of the wireless accelerometer signal could be nurse's station or, if in a hospital setting, a bedside aural alarm.

While various preferred embodiments have been disclosed herein showing use of an eyeglass head band retainer, and a medical device head band retainer, in another preferred embodiments, the head band device could be used to hold other apparatus in position on a wearer's head, or to monitor other medical data of a patient.

The above detailed description teaches certain preferred embodiments for the present inventive eyeglass and medical device retainer using a head band and loop elements. While preferred embodiments have been described and disclosed, it will be recognized by those skilled in the art that modifications and/or substitutions are possible and such modifications and substitutions are within the true scope and spirit of the present invention. It is likewise understood that the attached claims are intended to cover all such modifications and/or substitutions.

What is claimed is:

1. An eyeglass retainer to be worn with eyeglasses, comprising:
a head band worn around the circumference of a wearer's head,
looping means having two ends such that the first end is attached to the headband and the second end wrapping around the eyeglasses,
attaching means incorporated into the second end of the looping means such that the second end of the looping means is removably attachable to the headband and thereby maintaining the eyeglasses in the position desired by the wearer.

2. The eyeglass retainer of claim 1, wherein the attaching means is a hook and loop material.

3. The eyeglass retainer of claim 1, wherein the attaching means is a snap device.

4. The eyeglass retainer of claim 1, wherein the attaching means is a button device.

5. The eyeglass retainer of claim 1, wherein the headband further comprises two ends permitting adjustment of headband on a user's head.

6. The eyeglass retainer of claim 5, further comprising hook and loop material on said headband two ends thereby permitting adjustment of headband on a user's head.

7. The eyeglass retainer of claim 1, wherein the headband is fabricated from a moisture absorbing material.

8. The eyeglass retainer of claim 5, wherein the headband material is washable.

9. The eyeglass retainer of claim 1, wherein the headband is fabricated from a terry cloth type material.

10. A medical device retainer, comprising:
a head band worn around the circumference of a patient's head;
at least one loop element having two ends such that said first end is attached to the headband and said second end wraps around said medical device to be held in position on a wearer's head;
attaching means incorporated into said second end of said at least one loop element such that said second end of said at least one loop element is removably attachable to said headband;
thereby maintaining said medical device in a position necessary for proper medical use.

11. The medical device retainer of claim 10, wherein said medical device is at least one oxygen tube placed in a patient's nostril.

12. The medical device retainer of claim 10, wherein the attaching means is hook and loop material.

13. The medical device retainer of claim 10, wherein the headband is fabricated from a sterile material for medical use.

14. The medical device retainer of claim 10, wherein the headband further comprises two ends permitting adjustment of headband on a patient's head.

15. The medical device retainer of claim 14, further comprising hook and loop material on said headband two ends thereby permitting adjustment of headband on a patient's head.

16. The medical device retainer of claim 10, further comprising at least one sensor to measure a patient's body temperature by sensing said patient's forehead temperature.

17. The medical device retainer of claim 10, further comprising at least one sensor to measure a patient's pulse by sensing said patient's temple area.

18. The medical device retainer of claim 10, further comprising at least one accelerometer to sense rapid accelerations due to an inadvertent fall.

19. The medical device retainer of claim 18, further comprising a transmitter to send a wireless signal and alarm to at least one remote recipient if an inadvertent fall is detected.
